(19) United States
(12) Patent Application Publication
(10) Pub. No.: US 2007/0122867 A1
(43) Pub. Date: May 31, 2007

(54) Medical device for monitoring blood phenylalanine levels

(76) Inventors: Richard D. Shunnarah, Atlanta, GA (US); Sheldon W. May, Atlanta, GA (US); Charlie D. Oldham, Marietta, GA (US); Veronica De Silva, Atlanta, GA (US)

Correspondence Address: GREENBERG TRAURIG, LLP, MET LIFE BUILDING, 200 PARK AVENUE, NEW YORK, NY, 10166, US

(21) Appl. No.: 10/552,953

(22) PCT Filed: Apr. 15, 2004
(86) PCT No.: PCT/US04/11706

§ 371 Date: May 25, 2006

Related U.S. Application Data
(60) Provisional application No. 60/462,887, filed on Apr. 15, 2003.

(51) Int. Cl. C12Q 1/30; C12M 1/34

(52) U.S. Cl. 435/27; 435/287.1

(57) ABSTRACT

A medical device adapted for the monitoring of blood levels of phenylalanine utilizing colormetric analysis, comprising a unit containing testing elements, or insertion means for receiving a substrate having a test biological sample thereon and a means on said device for displaying a test result for a level of phenylalanine in the biological sample.

### FIELD OF THE INVENTION

The present invention relates to a medical device for monitoring blood phenylalanine with respect to PKU management and treatment.

### BACKGROUND OF THE INVENTION

Phenylketonuria (“PKU”) is a metabolic genetics disorder characterized by the inability of the body to utilize the essential amino acid, phenylalanine. Individuals with PKU accumulate too much phenylalanine, which is one of the amino acids found in protein-containing foods. For unknown reasons, an excess of phenylalanine in an infant's body is harmful to the development of the brain causing mental retardation unless treated during early infancy. When a very strict diet low in phenylalanine is initiated early and well maintained, individuals diagnosed with PKU can expect normal development and a normal life span. Treatment consists of lifelong dietary management and counseling, as well as continued blood phenylalanine monitoring.

PKU is caused by mutation in the gene that alters the function of the enzyme phenylalanine hydroxylase (PAH). This enzyme would normally convert phenylalanine to the amino acid tyrosine. In those individuals with PKU, the failure of the conversion results in a buildup of phenylalanine. Through a mechanism that is not well understood, the excessive amounts of phenylalanine is toxic to the central nervous system and causes the severe problems associated with PKU. Damage to the brain causes marked mental retardation by the end of the first year of life. Older children may develop movement disorders. Symptoms can include skin rashes, hyperactivity, mental retardation, seizures, microcephaly, speech delays, tremors, behavior abnormalities, delayed mental and motor skills, an offensive odor to sweat an urine, light coloration (complexion, hair and eyes).

In those individuals diagnosed with PKU, each will have varying amounts of enzyme deficiency. Some individuals have enough enzyme activity that the diet can be extensive, while others may have a very strict diet. The healthcare professionals at a PKU treatment program must determine the nature of the diet for an individual diagnosed with PKU.

In 2000, the National Center for Health Statistics reported 4,058,814 births in the United States. At the incidence rate of 1:10,000 approximately 405 births were diagnosed with PKU in the United States in 2000. It is estimated that approximately 14,000 individuals as infants, adolescents and adults reside in the United Sates diagnosed with PKU.

PKU is a genetic inborn error of metabolism that is detectable during the first days of life with appropriate blood testing via newborn screening. Universal screening of newborn babies began in the United States approximately 40 years ago with the discovery of the cause of PKU and the blood test designed to detect this metabolic genetics disorder. Dr. Robert Guthrie of the University of Buffalo developed the newborn screening test for PKU in 1961. Massachusetts became the first state to mandate screening for a genetic disorder in 1963. The Guthrie test for PKU is now mandated by all 50 states and the District of Columbia. Early screening, special diets and continued blood monitoring have allowed these children to grow normally and lead full and productive lives.

Most, if not all infants born with PKU will develop mental retardation without treatment. Treatment of those affected with PKU includes a strict diet regimen that is low in, or free of phenylalanine, particularly when the child is growing. To prevent mental retardation, treatment must begin in early infancy to ensure normal mental development. As a result of the problems associated with the discontinuation of the diet, it is believed that the diet, as well as the treatment regimen, should be maintained for life.

The treatment of PKU is complex, requiring routine collection of blood samples, maintenance of a highly restrictive diet, recording of food intake, and visits to a PKU treatment program. In the United States, each state screens the blood phenylalanine level of all newborns within the first days of life.

The goal of PKU treatment is to maintain a blood phenylalanine level between 2 and 10 mg/dL (120-600 micromol/L). Frequent monitoring of blood phenylalanine levels is of paramount importance, especially during the early years of life; with less frequent monitoring as age increases.

The frequency of blood phenylalanine monitoring will vary according to the individual's needs. “Development reliable home-testing is recommended, as well as measures to increase adherence.”(NIH Consensus Statement on Phenylketonuria: Screening and Management, October 2000).

There are numerous obstacles to overcome in the blood drawing necessary for the monitoring of phenylalanine levels in those individuals affected with PKU. Especially in children, blood drawing can be a hectic and frustrating experience for both the patient as well as the person involved with the blood draw. Preparing a child for a blood draw, gathering of needed materials including lancets, anesthetic creams, alcohol pads, Band-Aids, filter paper, address labels/envelopes for mailing, etc., is difficult at best. In addition, waiting times for test results can delay needed changes in treatment regimens. The constant needs and concerns for obtaining blood samples and sending the samples to a laboratory or the constant travel and difficulties inherent in those travels provides a unique need in a highly specialized marketplace for a medical device for monitoring blood phenylalanine.

In the last several decades analytical and clinical chemistry has developed to the point where many useful analytical measurements can be made using relatively simple and inexpensive instrumentation and often by unskilled personnel. Some of these technologies have now become over the counter, readily available, kits and instruments for home use. The most common of these is quantitative glucose measurement, used regularly by millions of Diabetics throughout the world. Using a microlancet to generate a small (50-100 microliter) blood droplet, the patient transfers the blood sample to a dip stick device, which serves to collect the sample, performs needed separation steps, and delivers the sample to one or more analytical zones in which a specific chemical reaction is carried out, resulting in a signal which is read by a small, inexpensive analytical instrument. In the case of diabetes, glucose specific dipsticks are used and small hand held reflectance colorimeters, commonly called Glucometers, are employed with acquisition costs in the range of $30-100 or more.

The individual Dipsticks cost in the range of 50 cents to $2.00, depending on the manufacturer and quantity considerations. In addition to the enzyme based colorimetric assays used for glucose, immunoassays can also be employed, the most common of which is the over the counter pregnancy test. The current over the counter cholesterol test is an enzyme based system.

The major Diabetes Control and Complications Trial (DCCT) recently documented the enhanced health benefits of tight glycemic control for diabetes. Regular monitoring of glucose and regulation of insulin intake leads to much more effective management of the disease and the minimization of the chronic complications which are so burdensome to both the patient and to the health care system.

The diabetes community is leading and driving major research and development activities to further improve the measurement and monitoring of glucose and of other metabolites important to diabetes, with an emphasis on sampling methods which do not involve the trauma and discomfort of blood sampling. There is a move towards the use of interstitial fluid as the analytical sample and even to the development of truly non-invasive methods of analysis. Considerable research and development is now being focused upon minimally invasive approaches for obtaining samples of interstitial fluids for glucose analysis. Such fluid can be collected from the skin epidermal layer, which is devoid of blood vessels or nerves. The process is therefore painless and bloodless.

The present invention utilizes the known technology for glucose testing adapted for QO PKU management, i.e., for phenylalanine blood level monitoring. It is contemplated that the next generation will utilize painless and bloodless technology.

It is an objective of the invention to provide a specialized blood monitoring product for use in metabolic genetic disorders. More specifically, it is an objective of the invention to provide a blood-monitoring product for measuring phenylalanine levels in those individuals affected with PKU who are under strict medical supervision. A still further objective is to provide a blood phenylalanine monitoring product for in-home use by those individuals affected with PKU.

### SUMMARY OF THE INVENTION

The present invention relates to a medical device and test strips to be read by the device similar to a glucose monitor and strips used by diabetics which enables individuals affected with PKU to routinely monitor blood phenylalanine levels at home on an as needed basis by the individual. The present strips and device routinely monitor blood phenylalanine levels as necessitated by the PKU treatment regimen. In a preferred embodiment the present device will further comprise a memory storage of blood phenylalanine results over a lengthy treatment regimen. An embodiment of the device is such that blood sample drops can be placed on test strips that will continue to be purchased on an as needed basis as deemed appropriate by the prescribing genetics physician.

### DETAILED DESCRIPTION OF THE INVENTION

The present invention provides a novel test strip for analysis of biological liquids for the level of phenylalanine in the biological fluid. The present test strip comprises at least two superimposed layers, desirably discrete, in intimate contact. Preferably, such test strips are formed prior to application of the biological liquid sample for analysis.

Stated more particularly, the present invention provides integral analytical strips composed of multiple, superposed layers which can provide quickly within the strip a highly quantitative, detectable change in response to the presence of phenylalanine in liquid applied to the strip. The strips of this invention can be used for diagnostic and monitoring purposes and include a sample spreading layer in fluid contact with a reagent layer. The sample spreading layer, synonymously referred to herein as a spreading layer or a metering layer, is capable of distributing or metering within the layer substance(s) including at least a component of a liquid sample applied to the strip or a reaction product of such a component to provide, at any given time, a uniform concentration of such substance at the surface of the spreading layer facing, i.e. closer to, the reagent layer. The applied sample need not be confined. In various preferred embodiments, the spreading layer can be isotropically porous; that is, it is porous in every direction within the layer. Reference herein to isotropic porosity identifies the fact of substantial porosity in all directions within the spreading layer. It will be understood that the degree of such porosity may be variable, if necessary or desirable, for example regarding pore size, percentage of void volume or otherwise. It shall be understood that the term isotropic porosity (or isotropically porous) as used herein should not be confused with the terms isoporous or ionotropic, often used with reference to filter membranes to signify those membranes having pores that are continuous between membrane surfaces. Likewise, isotropic porosity should not be confused with the term isotropic, used in contradistinction to the term anisotropic, which signifies filter membranes having a thin “skin” along at least one surface of the membrane. See for example, Membrane Science and Technology, James Flinn ed, Plenum Press, New York (1970).

The reagent layer is a layer containing at least one material that is interactive with phenylalanine or a precursor of a reaction product of phenylalanine, and within which a change can be produced by virtue of such interactive material. The reagent layer is preferably of substantially uniform permeability to at least one substance spreadable within the spreading layer or a reaction product of such a substance. Uniform permeability of a layer refers to permeability such that, when a homogeneous fluid is provided uniformly to a surface of the layer, identical measurements of the concentration of such fluid within the layer, but made through different regions of a surface of the layer, will yield substantially equal results. By virtue of uniform permeability, undesirable concentration gradients within, for example, a reagent layer as described herein, are avoided.

Reference herein to fluid contact between a spreading layer and a reagent layer in an integral analytical element identifies the ability of a fluid, to pass in such test strip between superposed regions of the spreading layer and the reagent layer. Stated in another manner, fluid contact refers to the ability to transport components of a fluid between the layers in fluid contact.

The test strips of this invention can be self-supporting or the spreading layer, reagent layer in fluid contact with the spreading layer and any other layers can be carried on a support, such as a support that can transmit electromagnetic radiation of one or more wavelengths within the region between about 200 nm and about 900 nm.

Przybylowicz, E. P. et. al. (U.S. Pat. No. 3,992,158 (1976)) described a thin film format methodology to measure analyte concentrations in the blood/serum via an enzymatic colorimetric assay. The contents of this patent is expressly incorporated herein by reference thereto. Preliminary studies have been carried out to employ this methodology for measuring L-Phe concentrations. The films comprise of several layers: the reagent layer, the spreading layer and the filtering layer. The reagent layer is made of a hydrophilic polymer such as, for example, gelatin or agarose containing the buffered enzymatic reagents, where the reaction, which signals the absence/presence of the analyte, takes place. The spreading layer made of; for example, cellulose acetate pigmented with titanium oxide assists in the uniform spreading of the analyte and serves as the reflection surface, which enables the quantitation of the colored reaction products via reflection densitometry. The filtering layer, made of cellulose acetate and diatomaceous earth, removes large proteins and blood cells from the analyte improving ease and accuracy of detection.

In an embodiment of the invention shown in FIG. 1, the analytical element is composed of a support 10 bearing a reagent layer 12 in fluid contact with a spreading layer 14 which can also serve the function of filtering and also may provide a suitably reflective background for reflection spectrophotometric detection through support 10. Alternatively, layer 14 may be such that it does not reflect and detection can be accomplished in the transmission mode. Layer 14 can be, for example, an isotropically porous blush polymer layer which has been coated or laminated over layer 12.

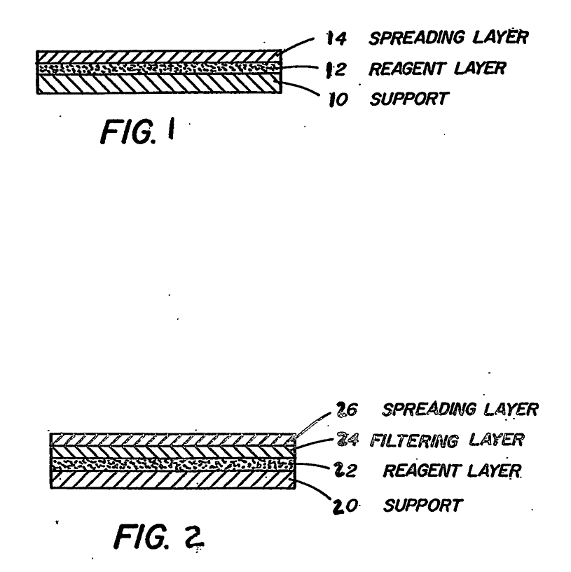

FIG. 2 illustrates a further embodiment of the invention is which the analytical element is composed of support 30, reagent layer 32, a filtering layer 34 can be formed from a semi-permeable membrane and which is in fluid contact with both layers 34 can be composed, for example, of titanium dioxide in blushed cellulose acetate.

The present home monitor for blood phenylalanine makes use of the enzyme phenylalanine dehydrogenase. As illustrated below, this enzyme converts phenylalanine to phenylpyruvate, with the concomitant production an equivalent amount of NADH. A colorimetric assay will then be used for detection of the NADH. For example, as illustrated below, NADH reduces a colorless tetrazolium compound to a colored compound that can be seen visually or measured by colorimetry. 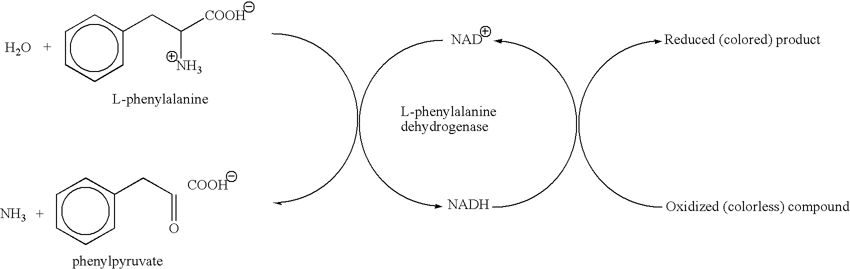
Oxidation of L-phenylalanine coupled to color formation
The NADH produced is measured using an election acceptor detection system.

The consensus ‘acceptable’ range for blood phenylalanine is 120 to 360 :moles/L. In practice, the upper limit is usually raised after five years of age to 480 :moles/L, and then is ‘allowed’ to go even higher after age ten if dietary compliance becomes an issue. There is also a need to monitor women during pregnancy.

The requisite limits of detection for a home monitor will be influenced by the sample volume available. The volume of blood from a finger stick is approximately 30 μL. Assuming a 30 μL drop of blood is used, the total amount of phenylalanine at the optimum lower limit of 120 μmoles/L is 0.60 μg, and at the optimum upper limit 360 μmoles/L is 1.8 μg. A lower limit of detection would need to be far enough below the 0.60 μg control value to accurately detect when the phenylalanine level is actually too low instead appearing to be low due to statistical variation between repeated measurements.

Several color reagents are useful and the limit of detection is influenced by the intensity of the color formed. Thionine, Rose Bengal, Methylene Blue, Azure C have been shown to react directly with NADH. Tetrazolium salts may also be used but may require an electron mediator such as 1-methoxy phenazine methosuflate.

### EXAMPLE

### Effect of Human Serum on the Enzymatic Colorimetric Assay

Human serum (type A/B, purchased from Sigma Aldrich) spiked with L-phenylalanine phenylalanine (L-Phe) solutions of various concentrations was used in colorimetric enzymatic assays. Control experiments carried out in the absence of serum but with equivalent L-Phe concentrations showed that the presence of serum caused a reduction in the rate of change of absorbance over time as well as a change in λₘₐₓ of the reduced dye.

Further investigations revealed that the presence of serum has a direct effect on the colorimetric portion of the assay by inhibiting the absorbance at 340 nm (λₘₐₓ, for NADH). The extent of inhibition varied depending on NADH concentration and amount of serum present in the assay mixture. Removal of proteins of MWs over 10,000 Da from the serum reduced the extent of inhibition to absorbance.

Experiments have been carried out to investigate whether a linear relationship between the rate of change of absorbance with time and L-Phe concentration could be obtained in the presence of serum.

A linear dependence was obtained upon increasing the enzyme concentration in the assay and narrowing the L-Phe concentration range used. In addition a greater consistency in collected data was obtained by filtering the serum through a 0.45 μm filter prior to use in the enzymatic assays.

A calibration curve was obtained for a L-Phe range of 0 to 200 μM, which corresponds to a 15 times dilution of serum and an undiluted L-Phe concentration range of 0 to 3000 μM.

Calibration Curve: 3 ml assay mixture contains 300 μM MTS, 150 μM PMS, 0.75 mM β-NAD^{+,} 0-200 μM L-Phe, 200 μl human serum and 0.17 u/ml L-Phe dehydrogenase in 5.4 mM potassium phosphate/43.5 mM triethanolamine buffer (pH 8.6).

[image]

The calibration curve, shown in above, is based on enzymatic colorimetric assays performed in at least triplicate, where the standard deviations range from ±0.0001 to 0.0002 for the rates of change of absorbance varying from 0.0026 to 0.005 AU/sec.

The accuracy of the calibration curves was tested with samples of unknown phenylalanine concentration (Calibration curve points denoted by hollow circles). The % error for predicting phenylalanine concentrations using this calibration curves ranged from 2 to 11%. The accuracy in predicting the unknown L-Phe concentrations was strongly dependent upon the enzyme concentration used in the assay.

Platelet-rich plasma (PRP) was prepared from human whole blood (stored in the presence of an anti-coagulant) by centrifugation at room temperature. PRP was stored in 1.5 ml aliquots at −20° C. and thawed in a 37° C. water-bath just prior to the start of the assay. Thawed PRP spiked with several L-Phe concentrations was used in the enzymatic colorimetric assay. Control experiments were carried out using serum spiked with equivalent L-Phe concentrations. The rates of change of absorbance over time at 520 nm were identical for serum and PRP-containing assay mixtures as shown in the table below:

Rate of Change of Absorbance with Time (AU/sec)

| | [L-Phe] = 0 μM | [L-Phe] = 75 μM | -L-Phe] = 125 μM | [L-Phe] = 250 μM |
|---|---|---|---|---|
| Serum | 0.0011 | 0.0023 | 0.003 | 0.0036 |
| Plasma | 0.0013 | 0.0022 | 0.0029 | 0.0036 |

Assay conditions: 3 ml assay mixture contains 300 μM MTS, 150 μM PMS, 0.75 mM β-NAD⁺, 0-250 μM L-Phe, 200 μl human serum or PRP and 0.25 u/ml L-Phe dehydrogenase in 5.4 mM potassium phosphate/43.5 mM triethanolamine buffer (pH 8.6).

Further experiments with a wider range of L-Phe concentrations were not carried out using PRP because of clotting problems. Upon obtaining more blood further options will be explored for storing and treating the plasma. For instance, PRP can be stored in the presence of sterile dextrose, which lessens fibrin formation or cryoprecipitated plasma can be prepared, which is deficient in several clotting factors. Overall, the manipulations to the plasma should be kept to a minimum since extensive blood treatment will not be performed at the home monitoring kit level.

Studies have been carried out to test a thin layer film format for the present test strips. Spectrophotometric measurements were carried out to show that the enzymatic assay reagents function in the presence of gelatin (the hydrophilic polymer) in aqueous format. The rate of change of absorbance was measured at 490 nm in a typical enzymatic colorimetric assay for several L-Phe concentrations in the presence of gelatin. The data below shows that the assay functions under the given conditions (the final pH of the assay mixture was 7.00, previous studies were carried out at pH 8.0):

| [L-Phe] (mM) | Rate of Change of Absorbance (AU/sec) |
|---|---|
| 0 | 0.0007 |
| 2.5 | 0.0014 |
| 5.0 | 0.0025 |

Assay conditions: 3 ml assay mixture contains 300 μM MTS, 150 μM PMS, 1.125 mM β-NAD⁺, 0-5000 μM L-Phe and 0.17 u/ml L-Phe dehydrogenase in 5.4 mM potassium phosphate/43.5 mM triethanolamine buffer (final pH 7.0).

Experiments were then carried out to demonstrate that the enzymatic assay reagents function after drying in gelatin format. A typical reagent mixture (containing gelatin, buffer, dye/mediator and enzyme/co-factor) was dried at room temperature under a steady stream of air in a 96-well micro plate. L-Phe solution (buffered at pH 8.6) was added to the dry reaction mixtures and absorbance changes monitored at 490 nm during a 20-minute using a Molecular Devices Microplate reader. The experiment clearly demonstrated that the reagents were active after a drying process in gelatin as the rate of change of absorbance in the presence of L-Phe was significantly higher as compared to the control mixture, which contained all reagents but no enzyme.

A preliminary calibration curve was obtained for a L-Phe range of 1 to 15 mM

[image]

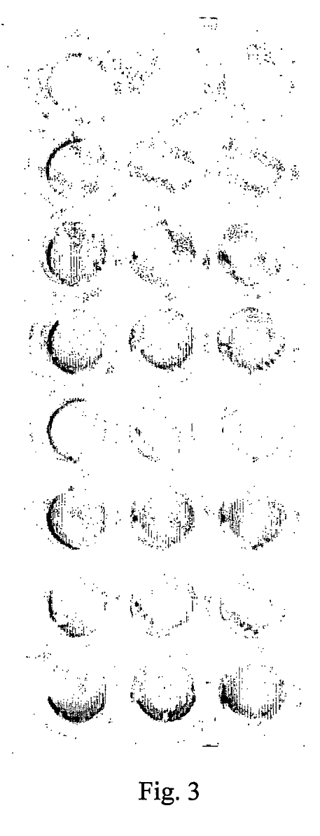

using the microplate assay described above (a snapshot of the microplate at end of the 20-minute reaction period is shown in the photograph of FIG. 3)

Assay conditions: approximately 65 μl assay mixture contains 75 μM MTS, 37.5 μM PMS, 0.375 mM β-NAD⁺, 0-15 mM L-Phe and 0.08 u/ml L-Phe dehydrogenase in 5.4 mM potassium phosphate/43.5 mM triethanolamine buffer (final pH 7.0).

The extent of stability of the enzymatic colorimetric reagents in dry gelatin format at room temperature using the microplate assay have been found stable over a period of 3 days.

Experiments have been carried out to make thin layer films, comprising the reagent, spreading and filtering layers. Gardo applicator rods and Bird type film applicators are employed to produce uniform layers of wet thickness ranging from 100 microM to 200 microM, which may become thinner upon drying (for example, a 100 microM gelatin film reduces to about a 20 microM thickness upon drying).” Layering of the reagent-containing gelatin mixture has been successfully accomplished.

The present device is primarily a home unit and is portable enough to take on trips. The design is a small device that uses an external power supply that plugged into the wall. It may be possible to further reduce the size to that similar to glucose monitors, if volumes will be lower for this product. Below is a subset of the requirements and the approach of the present design.

Hand-Held and Portable for In-Home Use:

The present device could be, preferably held in the hand but it works better if it is set on a table/counter top. A commercial-off-the-shelf plastic enclosure can be used for the initial designs. This could also be used for full production. The enclosure selected does have an option for a battery compartment but while the slides are used it can be kept as a table/counter top appliance.

Battery Powered—Rechargeable:

The present device can be plug-in or can be battery powered and preferably rechargeable. The unit can have a small external power pack that looks like a battery charger. The power circuits can be optimized, battery charging, added as well as space for batteries. The tester plus external power pack will still be together quite portable. The present device can also be fuel-cell powered.

The design will utilize a case having, for example, a common flat surface with a slanted display area. The standard enclosure is available in various colors including Bone (off white) and Black.

Ease of Use:

The unit itself can have three buttons, a place for the sample slide, a display, and two jacks (one for the power and one for a data connection). Pressing any of the three buttons will turn on the unit, and the display will then define the function of the buttons as they are used.

Software for the unit will run on a microprocessor chosen for its ease of programming and ease of incorporation into supporting circuits. This can be, for example, the microprocessor from the RCM3410 RabbitCore from Rabbit Semiconductor. Also included are a Xilinx low-power CoolRunner CPLD (complex programmable logic device) for glue logic. This facilitates recognizing button pushes when the unit is in a low-power, waiting mode.

A commercial-off-the-shelf display can be used. The display selected will be based on size, functionality, power consumption, cost, and availability.

It is understood that the display panel should show blood measurements in micromoles/liter, as well as milligram/deciliter. (One mg/dL=60 μmole/L).

The device will preferably weigh less than 1 pound.

The initial unit can have a FLASH memory storage file system that will allow the storage of the operating program, the optional set-up data, and the diary of the readings. An on-board watch-like battery will keep the contents of the memory safely stored when the power pack is not plugged in.

The only limitation on number of tests/hour is the amount of time it takes to perform the testing. The files will be maintained by date and time so they are automatically entered as the test is performed. Throughput should preferably be no more 10 tests/hour.

The variation in the reported results is more dependent on the strip than the electronics. The calibration routine will handle the variability of the measurements based on the electronics.

The planned unit is 3.6″ wide, 5.75″ deep, and approximately 2″ tall. The height of the initial units may be a little taller to allow quicker development, testing, and tweaking of the algorithms.

The weight of the unit should be in the 12-ounce range. The separate power supply, which plugs into the wall, will be similar to that used to charge a cell phone.

The test will be run from the main menu that is presented at start up so the test will require the minimum number of steps (for the operator). The data will be automatically filed to eliminate operator steps.

The system is designed to handle the strips of the present invention but it will be easy to change to another kind of test strip with minimal or no impact to the mechanical and functional design of the unit.

The current design uses the display to inform the operator of a success and the resulting reading or an error flashing on display. No audio feedback is currently designed into the unit but if that becomes required it can be easily added.

Results Can be Obtained in 10 Seconds or Less:

Data port to download results will be provided.

Memory capability of 100 test results will be provided.

1. A test element for determining the level of phenylalanine in a blood sample said test element comprising a layer onto which the sample of blood is applied and a reagent layer containing a material that is interactive with phenylalanine or a precursor of a reaction product of phenylalanine, wherein the reagent layer comprises a) an enzyme which converts phenylalanine to phenylpyruvate; b) buffered enzymatic colori-metric reagents wherein a reaction signals the presence/absence of phenylalanine; and c) a hydrophilic polymer.

2. (canceled)

3. The test element of claim 1, wherein the reagent layer comprises phenylalanine dehydrogenase.

4. (canceled)

5. (canceled)

6. The test element of claim 1, wherein the hydrophilic polymer is gelatin or agarose.

7. The test element of claim 1, wherein the colorimetric reagent is thionine, Rose Bengal, Methylene Blue, Azure C or a tetrazolium salt.

8. A medical device adapted for the monitoring of blood levels of phenylalanine utilizing colormetric analysis, comprising a unit containing testing elements according to claim 1, or insertion means for receiving the test elements according to claim 1 having a test blood sample thereon and a means on said device for displaying a test result for a level of phenylalanine in the blood sample.

9. The device of claim 11, further comprising a memory means for storing previous blood sample results and means for displaying the stored blood sample result.

10. (canceled)

11. A method of determining the presence or absence of phenylalanine in a blood sample comprising applying the blood sample to a test strip according to claim 1 allowing the blood sample to react with the reagent layer and colorimetrically determining a level of phenylalanine in the blood sample.

12. The device of claim 11, wherein the device is a plug-in device.
